(19) United States
(12) Patent Application Publication
(10) Pub. No.: US 2022/0331322 A1
(43) Pub. Date: Oct. 20, 2022

(54) COMPOUND FOR INHIBITING CELL DEATH

(71) Applicant: Christian-Albrechts-Universität zu Kiel, Kiel (DE)

(72) Inventors: Stefan Krautwald, Flintbek (DE); Ulrich Kunzendorf, Kiel (DE)

(21) Appl. No.: 17/641,670

(22) PCT Filed: Sep. 9, 2020
(86) PCT No.: PCT/EP2020/075134

§ 371 Date: Mar. 9, 2022

(30) Foreign Application Priority Data
Sep. 9, 2019 (EP) 19196185.3
Mar. 30, 2020 (EP) 20166882.9

(51) Int. Cl. A61K 31/513; A61P 37/06

(52) U.S. Cl. CPC A61K 31/513; A61P 37/06

(57) ABSTRACT

The present invention relates to the field of diseases or conditions that involve a pathologic level of RIPK1-dependent cell death. Specifically, the present invention refers to the use of the compound primidone or a pharmaceutically acceptable active metabolite, derivative, salt or solvate thereof for treating a disease or condition that involves a pathologic degree of RIPK1-dependent cell death. In a further aspect, the present invention provides a pharmaceutical composition comprising primidone or a pharmaceutically acceptable active metabolite, derivative, salt or solvate thereof for treating a disease or condition that involves a pathologic degree of RIPK1-dependent cell death.

The present invention relates to the field of diseases or conditions that involve a pathologic level of RIPK1-dependent cell death. Specifically, the present invention refers to the use of the compound primidone or a pharmaceutically acceptable active metabolite, derivative, salt or solvate thereof for treating a disease or condition that involves a pathologic degree of RIPK1-dependent cell death. In a further aspect, the present invention provides a pharmaceutical composition comprising primidone or a pharmaceutically acceptable active metabolite, derivative, salt or solvate thereof for treating a disease or condition that involves a pathologic degree of RIPK1-dependent cell death.

### BACKGROUND OF THE INVENTION

Necroptosis, a regulated form of necrosis, is a caspase-independent programmed cell death mechanism. Necroptosis is mediated by receptor-interacting protein kinase 3 (RIPK3) activation and the pursuant RIPK3-mediated phosphorylation of its pseudokinase substrate mixed lineage kinase domain-like protein (MLKL) (Cho et al., 2009; Sun et al., 2012). This initial stimulus prompts a conformational change that results in MLKL oligomerization, plasma membrane translocation, and lethal permeation of the lipid bilayer, leading to the release of cellular content, which triggers an inflammatory response.

The signaling pathways of various subtypes of regulated cell death have been analysed in detail in recent years. Another protein, receptor-interacting protein kinase 1 (RIPK1), appears to play a crucial role in the activation of regulated cell death, and it has been suggested that the inhibition of this protein might offer therapeutic benefit in a number of diseases which are characterized by a pathogenic level of apoptosis and necroptosis, such as ischemia-reperfusion injuries like myocardial infarction and acute kidney injury, neurodegenerative disorders like Parkinson's and Alzheimer's disease as well as stroke, sepsis and cancer. Although RIPK1 is currently considered as an important drug target by the pharmaceutical industry, there is so far no approved therapeutic drug that would allow inhibiting RIPK1-dependent apoptosis and necroptosis, thereby reversing or ameliorating the symptoms of the aforementioned diseases.

Against this background, it is an objective of the present invention to provide pharmaceutically active compounds and compositions that effectively inhibit RIPK1-mediated cell death and allow for the treatment of a disease or condition that involves a pathologic level of cell death. According to the invention, this objective is achieved by the compounds and pharmaceutical compositions defined in the enclosed claims.

The present invention is based on the surprising insight that the well-known anticonvulsant compound primidone is able to block RIPK1-dependent apoptosis and necroptosis by inhibiting RIPK1 activation. Accordingly, primidone and its active metabolites, derivatives, salts and solvates will be highly useful for blocking cell death in diseases or conditions which are associated with a pathological level or activation of RIPK1-dependent cell death.

### DESCRIPTION OF THE INVENTION

Activating necroptosis is known to require the activity of receptor-interacting protein 1 (RIPK1), a protein which mediates the activation of the two proteins RIPK3 and MLKL, both of which are critical downstream mediators of necroptosis. Owing to its central function in the necroptosis pathway, RIPK1 inhibitors have been suggested as useful therapeutics that are suitable to prevent the pathologic level of cell death which occurs in a number of diseases.

The present invention contemplates the use of the compound primidone for treating a disease or condition that involves a pathologic level or activation of RIPK1-dependent cell death. It was found in the course of this invention that primidone effectively inhibits RIPK1 activity in cells and tissues. As such, primidone is suitable for preventing or inhibiting RIPK1-dependent cell death, in particular apoptosis and necroptosis, in numerous diseases and conditions, including ischemia-reperfusion injury, neurodegenerative diseases like Parkinson's or Alzheimer's disease and sepsis.

Primidone is an anticonvulsant of the barbiturate class that is approved for a long time for therapeutic treatment of seizures, including partial and generalized seizures, and tremors. It is sold, amongst others, under the trade names Lepsiral®, Mysoline®, Resimatil®, and Liskantin®. Primidone is also known as desoxyphenobarbital or desoxyphenobarbitone. The IUPAC name of primidone is 5-Ethyl-5-phenyl-1,3-diazinane-4,6-dione. It was found herein that primidone prevents the activation of RIPK1 that normally occurs by (auto)phosphorylation. In the absence of phosphorylated RIPK1, the assembly of the necrosome consisting of the proteins RIPK1, RIPK3 and MLKL cannot occur. This ultimately leads to the prevention of RIPK1-mediated cell death.

In a first aspect, the present invention relates to the compound primidone or a pharmaceutically acceptable active metabolite, derivative, salt or solvate thereof, or a pharmaceutical composition comprising any of those, for use in a method of treating a disease or condition that involves a pathologic level of RIPK1-dependent cell death.

In one preferred embodiment, the disease to be treated is selected from the group consisting of reperfusion a injury disease, a systemic inflammatory disease, a transplantation-related disease, a neurodegenerative disease, an autoimmune disease, an ophthalmologic disease, a pulmonary disease, and a RIPK-expressing tumor disease.

In one embodiment, the disease or condition to be treated that involves a pathologic level of RIPK1-dependent cell death is a reperfusion injury disease. As used herein, a “reperfusion injury disease” includes diseases and conditions that involve tissue damage caused by reperfusion, i.e. tissue damage that occurs when the blood supply to tissue is restored after a period of ischemia or lack of oxygen. Tissue damage occurring after reperfusion is known to involve RIPK1-dependent processes. The importance of RIPK1-dependent cell death following ischemia and reperfusion has been demonstrated in rodent models (Linkermann et al. (2012)). Accordingly, it has been suggested that blocking RIPK1 activity could be a viable strategy for reducing ischemia reperfusion-related cell death in reperfusion injuries of the brain, retina, heart, kidney, and liver.

In one embodiment, the reperfusion injury disease to be treated is stroke. One of the main events occurring during ischemic brain stroke is cell death (Liu C. et al. (2017)). It is well-known that necroptosis mediated by RIPK and MLKL proteins contributes to brain injury after ischemic stroke. Therefore, primidone can effectively protect brain tissue from ischemic injury by blocking RIPK1-dependent necroptosis.

In another embodiment, the reperfusion injury disease to be treated is myocardial infarction (heart attack). Regulated necrosis is one of the main forms of cardiomyocyte death in heart disease and heart failure. Especially RIPK1-mediated necroptosis has been implicated in the progression of myocardial infarction (Oerlemans M I, et al. (2012)). Therefore, RIPK1 might serve as a novel therapeutic target for prevention of myocardial ischemia-reperfusion injury.

In yet another embodiment, the reperfusion injury disease to be treated is acute kidney failure Linkermann et al. (2012) or acute liver failure (Takemoto K. et al (2014)).

In another embodiment, the disease or condition to be treated that involves a pathologic level of RIPK1-dependent cell death is a systemic inflammatory disease which is preferably selected from the group consisting of sepsis and systemic inflammatory response syndrome (SIRS). Cell death mechanisms play an important role in the pathogenesis of these diseases. For this reason, the use of RIPK1 inhibitors for blocking RIPK1-dependent cell death has been considered particularly useful for treating sepsis (Degterev A. et al. (2019); Zelic M. (2018)).

In yet another embodiment, the disease or condition to be treated that involves a pathologic level of RIPK1-dependent cell death is a transplantation-related disease, such as graft-versus-host-disease. Cell death processes like apoptosis and necroptosis were found to contribute to organ failure during organ transplantation and in graft-versus-host-disease (Shi, S. et al. (2019), Falcon, C. et al. (2017); Kanou T. et al. (2018); Pavlosky A (2014)).

In another preferred embodiment, the disease to be treated that involves a pathologic level of RIPK1-dependent cell death is a neurodegenerative disease selected from Alzheimer's disease, Parkinson's disease, Huntington's disease, amyotrophic lateral sclerosis (ALS), traumatic brain injury, and multiple sclerosis (MS). It is well known that RIPK1-dependent necroptosis promotes cell death and neuroinflammation in the pathogenesis of these diseases (Yuan, J. et al. (2019)).

In yet another embodiment, the disease to be treated that involves a pathologic level of RIPK1-dependent cell death is an autoimmune disease. RIPK1-dependent cell death was found to be involved in the pathomechanisms that result in autoimmune disease like ulcerative colitis, Crohn's disease, rheumatoid arthritis, autoimmune cardiomyopathy, autoimmune hepatitis, lupus erythematosus, Graves' disease, Guillain-Barré syndrome (GBS), Hashimoto's thyroiditis, idiopathic thrombocytopenic purpura, juvenile idiopathic arthritis, myasthenia gravis, pemphigus vulgaris, psoriasis, Reiter's syndrome, scleroderma, Sjögren's syndrome, vasculitis, vitiligo, and Wegener's granulomatosis (Degterev A. et al. (2019)).

In yet another embodiment, the disease to be treated is an ophthalmologic disease, such as retinal degeneration or retinitis pigmentosa (Sato K. et al. (2013); Do Y J et al. (2017)).

In yet another embodiment, the disease to be treated is a pulmonary disease, such as chronic obstructive pulmonary disease (COPD) or acute respiratory distress syndrome (ARDS). It is particularly preferred that the disease to be treated is ARDS, preferably virus-induced ARDS. ARDS is a life-threatening complication that is associated with acute damage to the lungs. In addition, it is often associated with multi-organ failure in SIRS. It may result from infection with a pulmonary virus, such as a pulmonary coronavirus (Khot and Nadkar (2020)). The recent COVID-19 outbreak has shown that many patients infected with Sars-CoV-2 develop ARDS, with a high number of fatal cases. It has been known in the art that RIPK1-dependent cell death plays an important role in the development of ARDS (Pan et al. 2016).

In addition, as shown in the below examples, it was found herein that a SARS-CoV-2 infection triggers RIPK1 activation in respiratory epithelial cells collected as throat smears of symptomatic patients who had tested positive for SARS-CoV-2 by PCR. Therefore, primidone or a pharmaceutically acceptable active metabolite, derivative, salt or solvate thereof can be effectively used for treating ARDS. In a particularly preferred embodiment, the disease to be treated is coronavirus-induced ARDS, such as ARDS caused by Sars-CoV-2-.

In yet another embodiment, the disease to be treated is a cancer disease resulting from a tumor that overexpresses RIPK1. For example, an upregulation of RIPK1 has been described in lung and pancreatic tumours (Gong Y. et al. (2019)). Thus, in a preferred aspect of the invention, the disease to be treated is a cancer disease resulting from a RIPK1-expressing lung or pancreatic tumor.

As used herein, primidone refers to the compound 5-Ethyl-5-phenyl-1,3-diazinane-4,6-dione having the following structural formula:

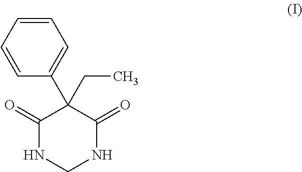 [SMILES: CCC1(c2ccccc2)C(=O)NCNC1=O]

Also included according to the invention are derivatives of the above structure (I) which are substituted or otherwise modified at one or several positions, as long as these modifications neither substantially affect the inhibitory effect of primidone on the RIPK1 activity nor lead to adverse results in terms of toxicity. For example, one or more hydrogen atoms of the C—H bonds of the heterocyclic ring systems can be substituted by halogen atoms, such as chlorine, bromine or iodine atoms. Further, the hydrogen of the C—H bonds can also be replaced by a short-chained alkyl group such as methyl, ethyl, propyl, or a long-chained alkyl group.

Active metabolites of primidone are also included by the invention. In particular, the invention also relates to the use of the primidone metabolite phenobarbital (PB), also known as phenobarbitone or phenobarb, for blocking RIPK1-dependent cell death. As used herein, phenobarbital refers to the compound 5-Ethyl-5-phenyl-1,3-diazinane-2,4,6-trione having the following structural formula:

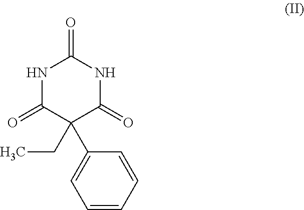 [SMILES: CCC1(c2ccccc2)C(=O)NC(=O)NC1=O]

The invention also refers to the use of the primidone metabolite phenylethylmalonamide (PEMA), for blocking RIPK1-dependent cell death. As used herein, PEMA refers to the compound 2-Ethyl-2-phenylmalonamide having the following structural formula:

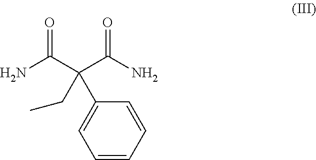

Also included according to the invention are derivatives of the above-depicted metabolite structures (II) and (III) which are substituted or otherwise modified at one or several positions, as long as these modifications neither substantially affect the inhibitory effect of the primidone metabolite on the RIPK1 activity nor lead to adverse results in terms of toxicity. For example, one or more hydrogen atoms of the C—H bonds of the heterocyclic ring systems can be substituted by halogen atoms, such as chlorine, bromine or iodine atoms. Further, the hydrogen of the C—H bonds can also be replaced by a short-chained alkyl group such as methyl, ethyl, propyl, or a long-chained alkyl group.

Pharmaceutically acceptable salts of primidone or its active metabolites or derivatives can also be used for treating the above diseases via RIPK1 inhibition. The term “pharmaceutically acceptable salt” refers to non-toxic acid addition salts, and alkali metal and alkaline earth metal salts, respectively. Exemplary acid addition salts include hydrochloride, hydrobromide, sulfate, bisulfate, acetate, oxalate, phosphate, citrate, maleate, fumarate, succinate, tartrate and lauryl sulfate. Exemplary alkali metal or alkaline earth metal salts include sodium, potassium, calcium and magnesium salts. In addition, ammonium salts and salts with organic amines can be used as well. Apart from the calcium salt, any other pharmaceutically acceptable cationic salt can be employed. The salts are, for example, salts that are obtained by reaction of the free acid form of primidone with a suitable base, such as sodium hydroxide, sodium methoxide, sodium hydride, potassium methoxide, magnesium hydroxide, calcium hydroxide, choline, diethanolamine, and others which are known in the prior art. Solvates of primidone or its metabolites are also part of the present invention. Solvates occur upon the addition of one or more solvent molecules to primidone or its metabolites. If the solvent is water, said addition is a hydration. Solvates of the contemplated active ingredient compound can be held together by ionic bonds and/or covalent bonds.

Primidone or the active metabolite, derivative, salt or solvate thereof will be formulated as a pharmaceutical composition suitable for administration to a subject, preferably a human subject. Pharmaceutical compositions comprising primidone can be formulated in accordance with standard methods using commonly known excipients. Such methods and suitable excipients and carriers are described, for example, in “Remington: The Science and Practice of Pharmacy”, Lippincott Williams & Wilkins; 21^{st} ed. (2005). Pharmaceutical compositions comprising primidone or an active metabolite, derivative, salt or solvate thereof may be formulated, for example, as compositions for oral, rectal, nasal or parental (including subcutaneous, intramuscular, intravenous and intradermal) administration. Depending on the target tissue to be treated, the compositions can occur in the form of granules, powders, tablets, capsules, syrup, suppositories, injection solutions, emulsions or suspensions.

Normally, the pharmaceutical compositions of the invention comprise primidone or an active metabolite, derivative, salt or solvate thereof in admixture with one or more pharmaceutically acceptable carriers which are physiologically compatible with the other ingredients in the compositions. The pharmaceutical compositions of the invention can also include further excipients, such as binders, diluents, dyes, sweeteners or the like.

Pharmaceutical compositions for oral, buccal, or sublingual administration may be provided as solid formulations, such as powders, suspensions, granules, tablets, pills, capsules or gel caps. Suitable carriers that are often used in solid compositions are discussed in “Remington: The Science and Practice of Pharmacy”, Lippincott Williams & Wilkins; 21^{st} ed. (2005) and comprise, for example, microcrystalline cellulose, methylcellulose, hydroxypropyl methylcellulose, casein, albumin, mannitol, dextran, sucrose, lactose, sorbitol, starch, agar, alginate, pectins, collagen, glycerides, or gelatine. Solid compositions for oral, buccal, or sublingual administration are may also comprise antioxidants, such as ascorbic acid, tocopherol or cysteine, lubricants, such as magnesium stearate, preservatives, such as paraben or sorbic acid, disintegrants, binders, thickeners, taste enhancers, dyes and the like.

Alternatively, pharmaceutical compositions for oral, buccal, or sublingual administration may also be provided as liquid formulations, for example, as emulsions, syrups, suspensions or solutions. These formulations can be prepared by admixing primidone or its active metabolite, derivative, salt or solvate with a liquid carrier which may be a sterile liquid, such as oil, water, alcohol or combinations thereof. Oils which are suitable for being used in liquid dosage forms comprise, for example, olive oil, sesame oil, peanut oil, rape oil, and corn oil. Suitable alcohols comprise ethanol, isopropyl alcohol, hexadecyl alcohol, glycerol and propylene glycol. If the pharmaceutical composition is formulated in the form of a suspension, a fatty acid ester, such as ethyl oleate or isopropyl myristate, a fatty acid glyceride, or an acetylated fatty acid glyceride may be added. Furthermore, substances like mineral oil or petrolatum are often added to suspensions.

It is preferred herein that the pharmaceutical composition of the invention is formulated for being administered by injection, e.g. by bolus injection or by continuous infusion. Compositions suitable for injection typically comprise an aqueous solution, an aqueous suspension or an oil suspension which has been prepared by use of a suitable solvent or suspending agent. Injectable compositions may also comprise stabilizers or surfactants that can optionally be added to these formulations. Injectable compositions may be formulated in the form of powders that can be reconstituted in a suitable solvent prior to use. Examples include, amongst others, lyophilized or spray-dried powders.

The route of administration which is most suitable for the respective therapy and the amount of the primidone compound to be administered can be determined by a skilled person using routine methods. Parameters which influence the amount of primidone or an active metabolite, derivative, salt or solvate thereof in the pharmaceutical composition to be administered include the type and severity of the disease, age, body weight, sex and overall state of health of the patient to be treated, the simultaneous administration of other therapeutic agents, and other parameters.

Suitable pharmaceutical compositions for the administration to humans will typically comprise 1 mg to 50 mg of the primidone compound per kilogram body weight of the patient. For children, primidone is typically administered at 1 mg to 30 mg of the primidone compound per kilogram body weight, preferably at 5 mg to 20 mg of primidone per kilogram body weight, and more preferably at 10 mg to 20 mg of primidone per kilogram body weight. For adults, primidone is typically administered at 1 mg to 20 mg of the primidone compound per kilogram body weight, preferably at 5 mg to 15 mg of the primidone compound per kilogram body weight, and more preferably at 10 mg to 15 mg of the primidone compound per kilogram body weight. Stated differently, the overall amount of the primidone compound administered to the patient daily typically is in the range from 5 mg to 5000 mg, preferably from 50 mg to 2500 mg, and more preferably from 500 mg to 1500 mg.

It is preferred that primidone or the active metabolite, derivative, salt or solvate thereof is administered in an amount that the concentration of the primidone compoin the plasma is between 0.1 μg/ml and 12 μg/ml, preferably between 0.5 μg/ml and 10 μg/ml, more preferably between 1 μg/ml and 7.5 μg/ml, and even more preferably between 2 μg/ml and 5 μg/ml. The therapy can be monitored and adapted accordingly to provide for such plasma concentrations.

The therapeutic effectiveness of the pharmaceutical compositions of the invention can be evaluated by using parameters known in the art. These parameters comprise, amongst others, the effectiveness of the composition according to the invention in eradicating symptoms of the treated disease, the response rate, the time until progression of the disease and the survival rate of the treated patients. Preferably, the compositions of the invention lead to a complete response in the patient. As used herein, complete response means the elimination of all clinically detectable disease symptoms as well as the restoration of normal results in blood count, x-ray examination, CT pictures, and the like. Such a response lasts preferably a month after the treatment has been stopped. The anti-compositions of the invention can also result in a partial response in the patient. In a partial response, the measurable tumor burden in the patient is reduced. At the same time, an improvement occurs in one or more symptoms which are caused by the disease, e.g. fever, loss of weight, and the like.

Primidone or the active metabolite, derivative, salt or solvate thereof can be used in combination with at least one additional therapeutic agent, such as another inhibitor of apoptosis or necroptosis, like Nec-1 or Nec1s. The primidone compound and the additional therapeutic agent can be administered to the subject in the form of a single pharmaceutical composition comprising both compounds agents, optionally in combination with suitable excipients and carriers. Administration of such a pharmaceutical composition will then automatically result in a simultaneous administration of the primidone compound and the additional therapeutic agent to the subject. Alternatively, the primidone compound and the additional therapeutic agent may also be administered separately from each other, i.e. in the form of two separate pharmaceutical compositions, one containing the primidone compound, and the other containing the additional therapeutic agent. The two separate compositions can be administered simultaneously or sequentially in either order to the same or different administration sites. For example, a combination therapy with the above-mentioned agents may begin at day 1 of a treatment period, and a first therapeutically effective dose of the primidone compound is administered at that day. A first therapeutically effective dose of the other therapeutic agent, such as another inhibitor of apoptosis or necroptosis, like Nec-1 or Nec1s, can be administered either on the same day, e.g. simultaneously or within about 30, 60, 90, 120, 150, 180, 210 or 240 minutes after administration of the primidone compound. Alternatively, the other therapeutic agent can be administered 2, 3, 4, 5, 6 or 7 days after administration of the primidone compound. A skilled person will readily be able to design administration regimens which are suitable for providing the maximum therapeutic effect.

### BRIEF DESCRIPTION OF THE FIGURES

FIG. 1 shows the results of stimulation assays demonstrating that primidone (referred to as compound A) blocks both RIPK1-dependent apoptosis and RIPK1-dependent necroptosis.

FIG. 2 shows the results of stimulation assays demonstrating that primidone not only blocks RIPK1-dependent cell death which is induced by ligation of death receptors like TNFR1 but also by endosomal Toll-like receptor 3 (TLR3) agonist which serves as a sensor of viral infection and sterile tissue necrosis.

FIG. 3 shows the results of stimulation assays demonstrating that primidone (referred to as compound A) does not repress NF-κB activation.

FIG. 4 shows the results of profiling kinase assay demonstrating that primidone (referred to as compound A), unlike Nec-1s, does not inhibit the kinase activity of RIPK1.

FIG. 5 shows the results of binding studies demonstrating that during TSZ-induced necroptosis RIPK1 binds to the TNF-α receptor in the presence of primidone.

FIG. 6 shows the results of stimulation assays demonstrating that during TSZ-induced necroptosis primidone prevents the phosphorylation (activation) of RIPK1, thereby inhibiting the assembly of the necrosome.

FIG. 7 shows the results of stimulation assays demonstrating that primidone specifically blocks RIPK1-mediated cell death.

FIG. 8 shows the results of stimulation assays demonstrating that primidone is longer active compared to Nec-1s.

FIG. 9 shows the results of stimulation assays demonstrating that both primidone metabolites, phenylethylmalonamide (PEMA) and phenobarbital (PB), respectively, block RIPK1-dependent cell death processes.

FIG. 10 shows the results of survival experiments in a SIRS mouse model demonstrating that primidone is able to protect against the lethal consequences of SIRS.

FIG. 11 shows the results of experiments made in a murine model of renal ischemia-reperfusion (IR). Vehicle-treated mice in the IR group had significantly higher plasma levels of serum urea (A) and creatinine (B) than primidone-treated mice. A TUNEL fluorescence assay (C) showed a significant reduced number of cells undergoing regulated cell death in animals treated with primidone.

### EXAMPLES

The present invention will be described in the following by preferred embodiments which merely illustrate the invention, but should by no means limit the invention.

### Example 1: Primidone Blocks RIPK1-Mediated Apoptosis and Necroptosis

Murine fibroblasts (L929 cells) were stimulated for 24 h at 37° C. in the presence of (a) vehicle, (b) 10 ng/ml tumor necrosis factor alpha (TNF-α), (c) 1 μM 5Z-7-oxozeaenol (5Z-7), (d) a combination of 10 ng/ml TNF-α and 1 μM 5Z-7, (e) a combination of 10 ng/ml TNF-α, 1 μM 5Z-7, and 20 μM necrostatin-1 (Nec-1s), (f) a combination of 10 ng/ml TNF-α, 1 μM 5Z-7, and 1 mM primidone (primidone is referred to in the Figures as compound A), (g) a combination of 10 ng/ml TNF-α, 1 μM 5Z-7, and 25 μM zVAD, (h) a combination of 10 ng/ml TNF-α, 1 μM 5Z-7, and 20 μM Nec-1s, and (i) a combination of 10 ng/ml TNF-α, 1 μM 5Z-7, 25 μM zVAD, and 1 mM primidone. TNF-α is a pro-inflammatory cytokine which triggers cell survival (canonical pathway) or cell death, depending on the cellular context. 5Z-7-oxozeaenol (5Z-7) is an inhibitor of transforming growth factor activated kinase 1 (TAK1). TAK1 is an intermediate in the signaling pathway of the TNF-receptor type 1 (TNFR1), and it is known that TAK1 is essential for the prevention of TNF-induced cell death. Cells in which TAK1 is disrupted or otherwise blocked are hypersensitive to TNF-α-induced cell death due to diminished prosurvival pathways including NF-κB and reduced antioxidant enzymes which results in activation of caspases. The compound zVAD is a pan-caspase inhibitor. Nec-1s is a Necrostatin-1 analogue with superior selectivity and stability which can be purchased, e.g. from Abcam (Berlin, Germany), and inhibits the kinase activity of RIPK1.

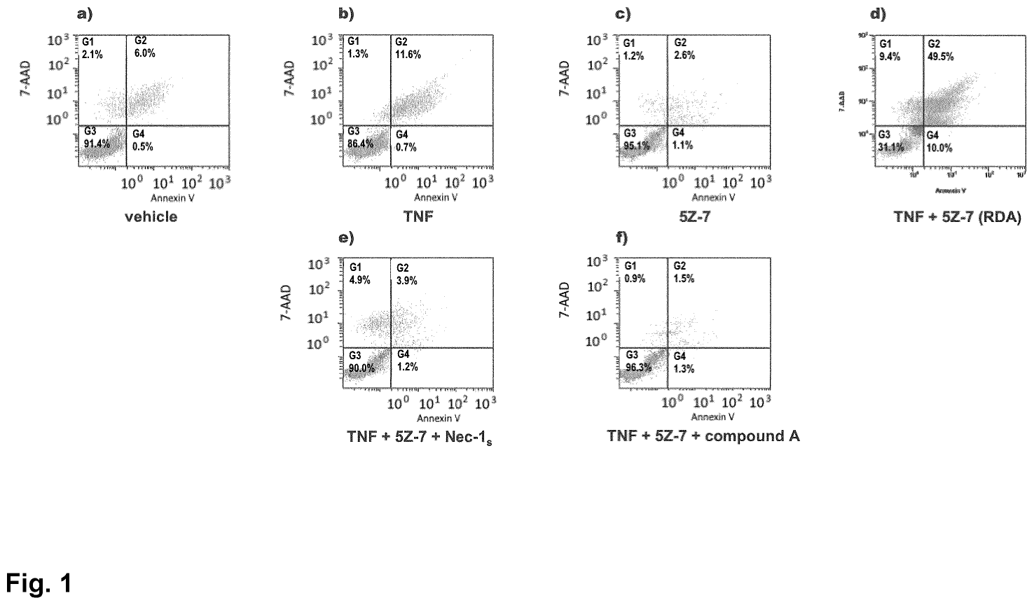

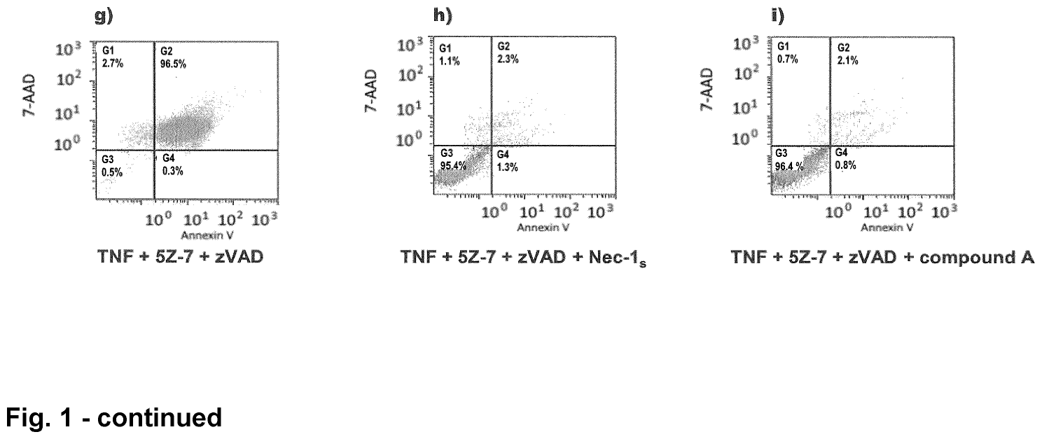

Results: As shown in panel (d) of FIG. 1, the combination of TNF-α and 5Z-7 induces RIPK1-dependent apoptosis (RDA). As this cell death is RIPK1-dependent, it can be blocked with the RIPK1 inhibitor Nec-1s, as depicted in panel (e). As shown in panel (f), RIPK1-dependent apoptosis could also be blocked with primidone. Since the cell death induced by TNF-α+5Z-7 is apoptotic, it should be possible to block it by adding a pan-caspase inhibitor like zVAD. It can be seen in panel (g) that cell death occurs, even though apoptosis is blocked which means that cell death shifts after inhibition of caspase-8 from apoptosis to necroptosis. This fact is confirmed in panel (h) showing that the addition of Nec-1s prevents RIPK-1 dependent cell death. Again, it was found that primidone was also able to block this RIPK-1-mediated necroptosis, see panel (i). In summary, this experiment shows that primidone can block both RIPK1-dependent apoptosis and RIPK1-dependent necroptosis.

### Example 2: Primidone Blocks TLR3-Mediated Cell Death

Cell death was induced by activation of the Toll-like receptor 3 (TLR3). TLR3 is mainly expressed on immune cells, where it senses pathogen-associated molecular patterns and initiates innate immune response. The TLR3 agonist poly (I:C) was developed to mimic pathogens infection and boost immune system activation. In experimental models, poly (I:C) is known to induce regulated cell death in cells expressing TLR3. To examine whether primidone also blocks TLR3-mediated cell death, murine L929 cells were stimulated for 24 h at 37° C. with vehicle (a), 1 μg/ml of the TLR3 ligand poly (I:C) alone (b) or in combination with 25 μM zVAD (c). In addition, cells were stimulated with 1 μg/ml poly (I:C), 25 μM zVAD and 20 μM Nec-1s (d) and with 1 μg/ml poly (I:C), 25 μM zVAD and 1 mM primidone (e).

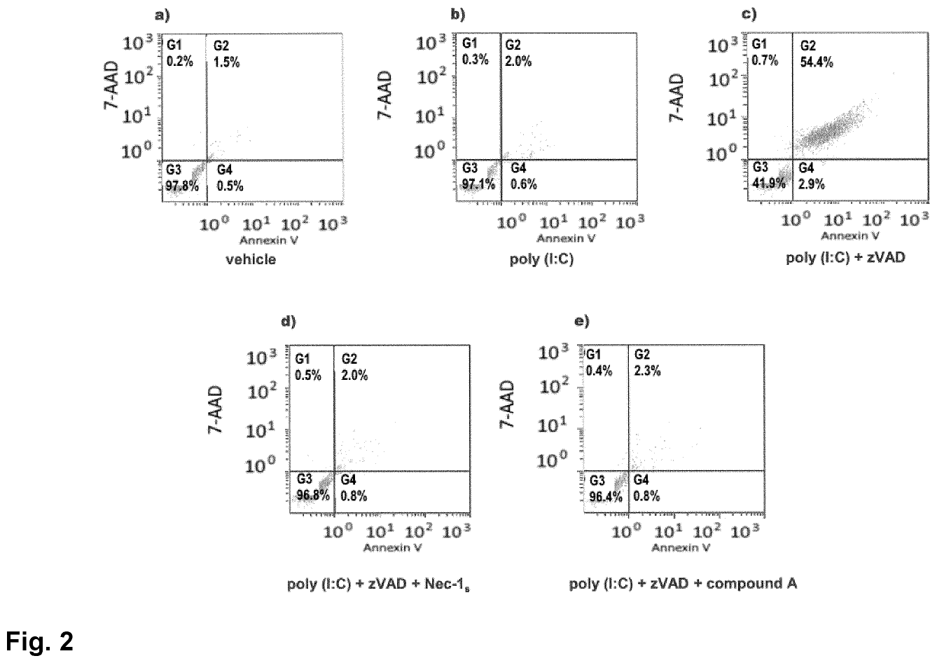

Results: As shown in panel (b) of FIG. 2, the addition of poly (I:C) alone does not induce cell death. Cell fate decisions following TLR signaling parallel death receptor signaling and rely on caspase-8 to suppress RIPK-dependent programmed necrosis. Therefore, the combined administration of poly (I:C) and zVAD induces a cell death, as shown in panel (c). Both Nec-1s and primidone effectively protect cells from this RIPK1-mediated cell death induced by poly (I:C) and zVAD, as shown in panels (d) and (e), respectively.

### Example 3: Primidone does not Repress the NF-κB Pathway

It was then analyzed whether primidone interferes with the canonical NF-κB signaling pathway which drives the expression of pro-survival molecules. The binding of TNF-α to its corresponding receptor (TNFR1) initially results in the activation of the NF-κB signaling pathway. In this early phase of TNF activation, the cells are protected from cell death by the presence of a membrane-bound complex known as complex I which is formed within seconds after engagement of TNFR1 by TNF-α. This complex induces the expression of pro-survival molecules via activation of the canonical NF-κB pathway. As the default response of most cells to TNF-α is survival and NF-κB-mediated upregulation of pro-survival genes, it was analyzed whether primidone functions in a survival-signaling mode and thus indirectly prevents cell death signaling. For this purpose, murine L929 cells were stimulated at 37° C. for different time periods (as indicated) with 100 ng/ml TNF-α+25 μM zVAD (TZ) in the absence (vehicle) or presence of 1 mM primidone. Subsequently, a Western blot analysis of the cell lysates using a specific p-NF-κB antibody was performed.

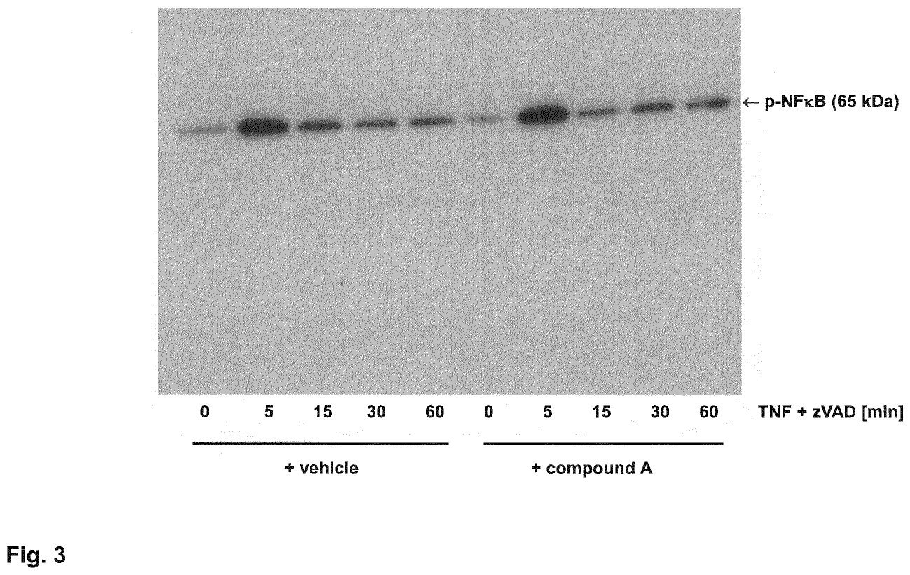

Results: Activation (phosphorylation) of NF-κB was detected 5 minutes after the treatment of the cells with TZ in the absence (vehicle) and in the presence of primidone (see FIG. 3), indicating that primidone does not inhibit or degrade any of the proteins required for the formation of complex I which would then prevent the formation of the cell death-inducing complex II.

### Example 4: Primidone does not Bind to the RIPK1 Kinase Domain

The kinase domain of RIPK1 is considered to have a key function in the signaling pathway. Known inhibitors of necroptosis like Nec-1s bind to the kinase domain of RIPK1, thereby interfering with the function of the protein. It was therefore investigated whether primidone binds to the kinase domain of RIPK1. To clarify, a KINOMEscan™ profiling kinase assay was performed. This assay measures the ability of compounds that bind to the kinase active site of RIPK1 to directly (i.e. sterically) or indirectly (i.e. allosterically) prevent kinase binding to an immobilized ligand. Dissociation constants (Kd) for test compound-kinase interactions are calculated by measuring the amount of kinase captured on the solid support as a function of the test compound concentration.

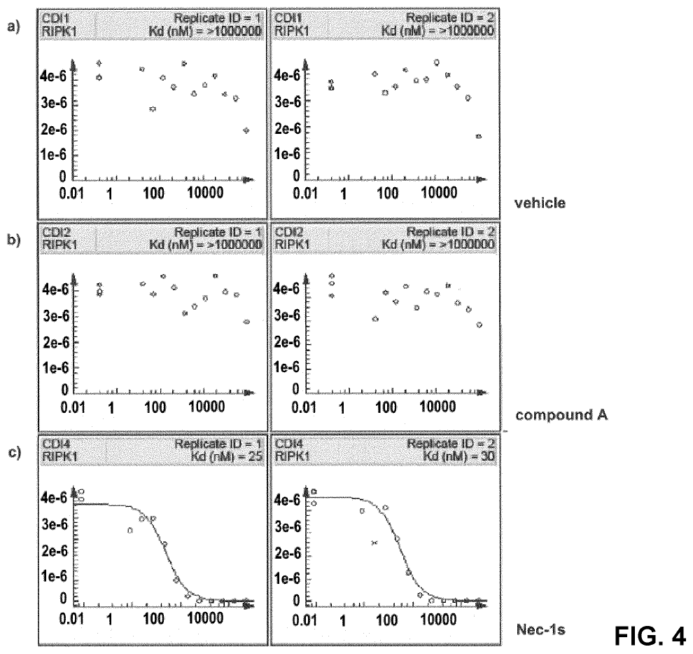

Results: It can be seen that Nec-1s, a compound that is known to bind to the kinase domain of RIPK1, prevents the kinase from binding to an immobilized ligand, as shown in row (c) of FIG. 4. It can be taken from the panels that the dissociation constant (Kd) value plotted on the ordinate decreases constantly with increasing Nec-1s concentration. Such a course cannot be observed for rows (a) and (b) which reflect the results from the kinase assay obtained with vehicle (a) or primidone (b), respectively. This means that primidone, unlike Nec-1s, does not inhibit RIPK1 by binding directly to its kinase domain.

### Example 5: Primidone does not Prevent Complex I Assembly

It was tested whether primidone interferes with binding of TNF-α to its corresponding receptor TNFR1. To induce cell death, human U937 cells were treated in the absence (vehicle) or presence of 1 mM primidone with 100 ng/ml Fc-tagged TNF-α+1 μM SMAC mimetic SM164+25 μM zVAD (TSZ) for different time periods, followed by immunopurification of the immunoprecipitated TNF-α-induced complex I using μMACS™ protein A/G microbeads, and western blot analysis using a specific RIPK1 antibody.

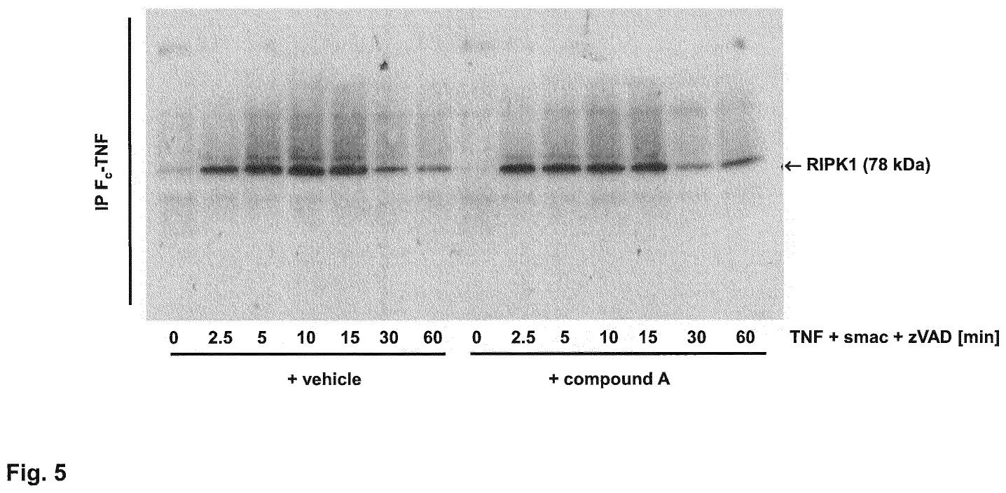

Results: As shown in FIG. 5, RIPK1 binds to TNFR1 regardless of whether primidone was present during stimulation or not. The right half of FIG. 5 shows the result in the presence of primidone, while the left half of FIG. 5 shows the result without primidone. This indicates that the TNF-α-induced formation of complex I is not influenced at this stage by the presence of primidone.

### Example 6: Primidone Prevents RIPK1 Activation

It was then analyzed if the previously immunoprecipitated RIPK1 is activated by phosphorylation. For this purpose, the Western Blot shown before (FIG. 5) was “stripped” by chemically removing all reagents from the blot and re-developed with an antibody that only detects RIPK1 in its activate (i.e. phosphorylated) form.

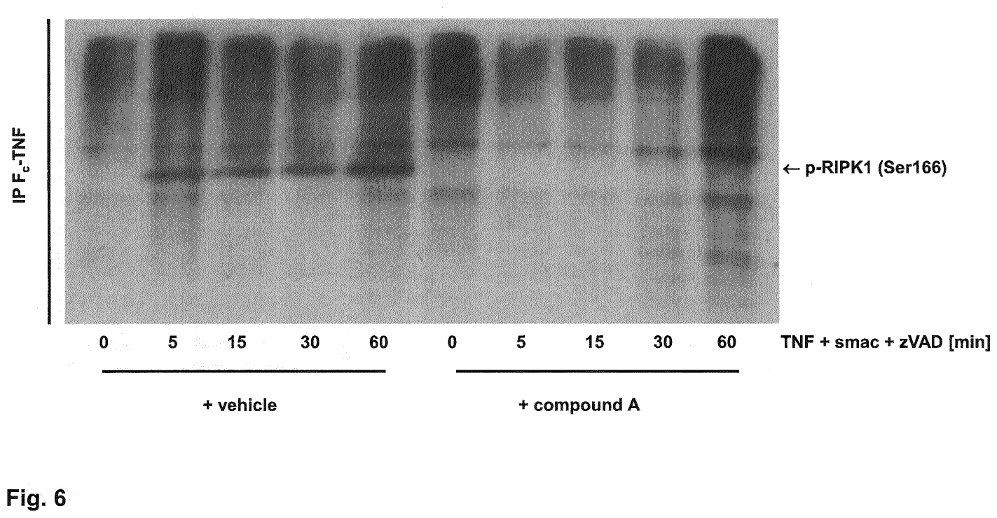

Results: It was found that TSZ treatment of the U937 cells resulted in a time-dependent activation (phosphorylation) of RIPK1 at residue Ser166 (left half of FIG. 6), which is completely blocked by the addition of primidone (right half of FIG. 6), indicating that primidone prevents the activation of RIPK1 which is a major function of RIP1 kinase in TNF-α-induced cell death.

### Example 7: Primidone Blocks RIPK1-Mediated Cell Death

Since primidone, amongst others, suppresses death receptor-initiated RIPK1-dependent signaling, it was tested whether primidone also inhibits death receptor-initiated processes that are not dependent on RIPK1. To this end, human T cells (Jurkat cells) were incubated for 5 h at 37° C. in the presence of (a) vehicle, (b) 5 ng/ml anti-Fas antibody, (c) 5 ng/ml anti-Fas antibody in combination with 25 μM of the pan-caspase inhibitor zVAD, and (d) 5 ng/ml anti-Fas antibody in combination with 1 mM primidone. The addition of the anti-Fas antibody induces apoptosis, a caspase-dependent form of regulated cell death that can be inhibited by the addition of the pan-caspase inhibitor zVAD.

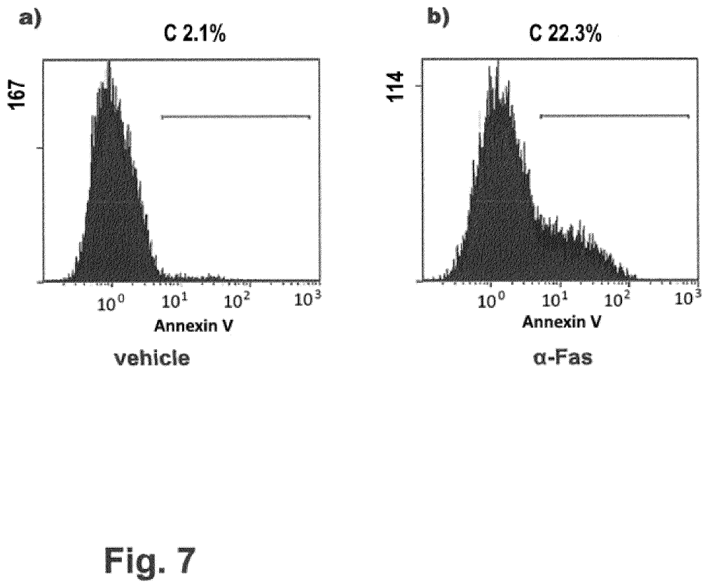

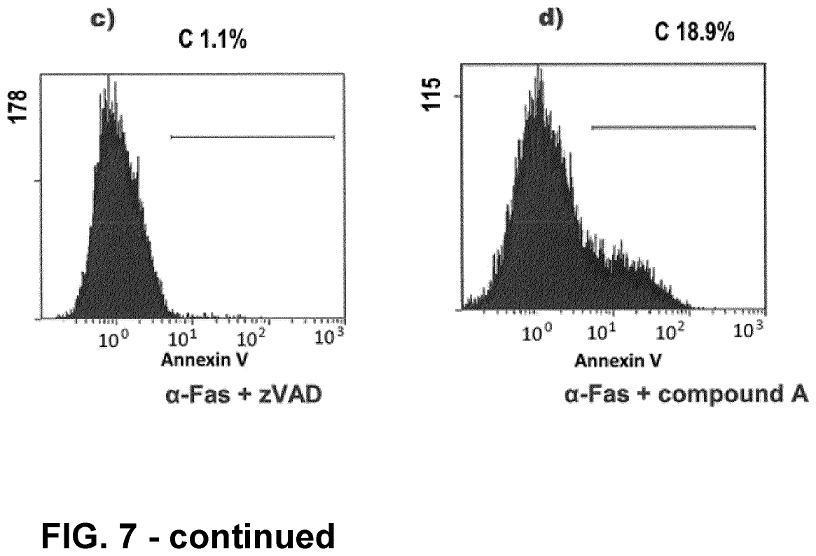

Results: The results are depicted in FIG. 7. Panel (a) expectedly shows no cell death in the presence of the negative control. Panel (b) demonstrates that the addition of an anti-Fas antibody induces cell death. Cell death induced in this way is caspase-dependent, but RIPK-1-independent. Accordingly, cell death can be completely blocked by the addition of the pan-caspase inhibitor zVAD, see panel (c). By contrast, the addition of primidone has no inhibitory effect as shown in panel (d). It follows from this experiment that primidone specifically blocks RIPK1-mediated cell death.

### Example 8: Primidone Shows Longer Activity than Nec-1s

The time profile of primidone for blocking cell death was compared to the one of Nec-1s. For this purpose, murine L929 cells were stimulated at 37° C. for 24 h with 10 ng/ml TNF-α+25 μM zVAD (TZ) in the presence of 1 mM primidone and 25 μM Nec-1s, respectively, for the indicated durations. Nec-1s and primidone were added 30 min before, 60 min and 180 min after the induction of RIPK1-mediated necroptosis, respectively. Cell death was quantified by FACS analysis using 7-amino-actinomycin D and phosphatidylserine accessibility (Annexin V staining) as markers.

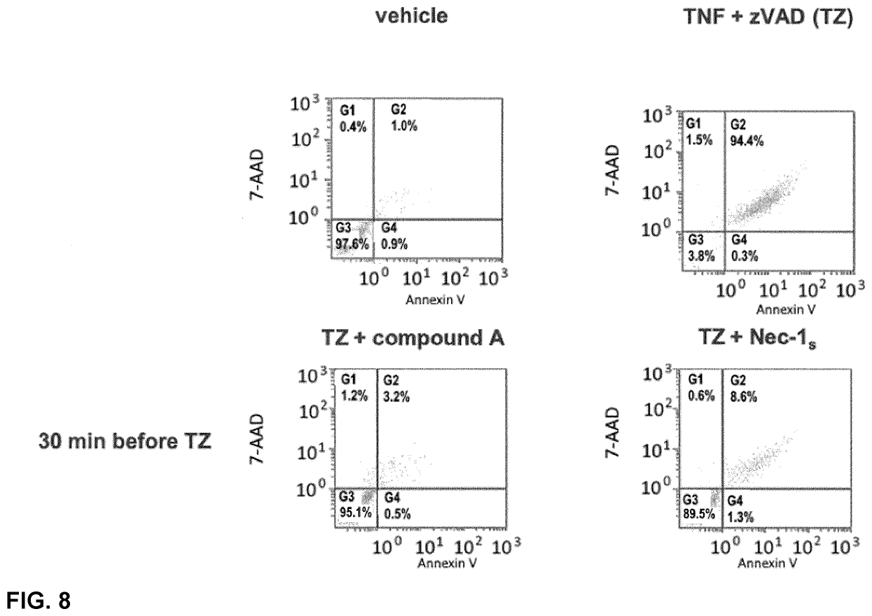

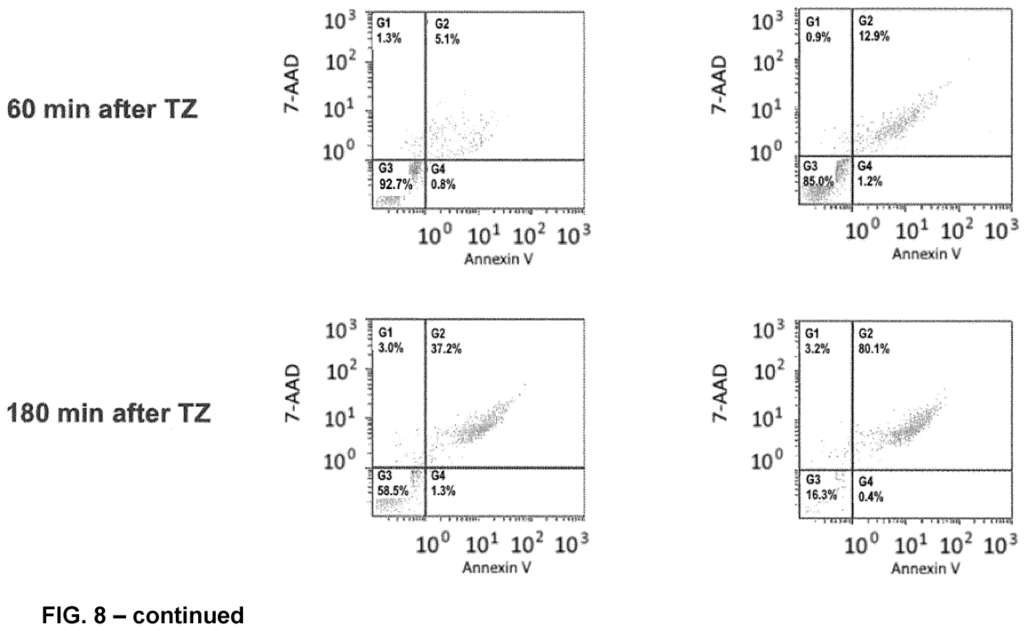

Results: The results are depicted in FIG. 8. Data of one representative experiment out of three independent experiments are depicted. It can be seen that 60 minutes after induction of cell death, no difference between Nec-1s and primidone can be detected in the efficacy profile. Both compounds still block cell death extremely effectively. In the sample containing primidone, 92.7% of the cells were still alive albeit primidone have been added one hour after induction of cell death. In the sample containing Nec-1s, 85.0% of the cells were still alive in this setting. Primidone, unlike Nec-1s, still protects significantly against cell death even if it was added 3 hours after stimulation of cell death. In the sample containing primidone, 58.6% of the cells were still alive even though primidone was added 3 hour after cell death induction. In the sample containing Nec-1s, only 16.3% of the cells were still in this setting. Therefore, it can be assumed that the longer lasting protective effect exerted by primidone is of enormous importance in everyday clinical practice.

### Example 9: The Metabolites PEMA and PB are Active as Well

Murine fibroblasts (L929 cells) were incubated for 24 h at 37° C. in the presence of (a) vehicle, (b) 1 mM PEMA or PB, respectively, (c) a combination of 10 ng/ml TNF-α+25 μM zVAD (TZ), and (d) a combination of TZ+PEMA or TZ+PB, respectively.

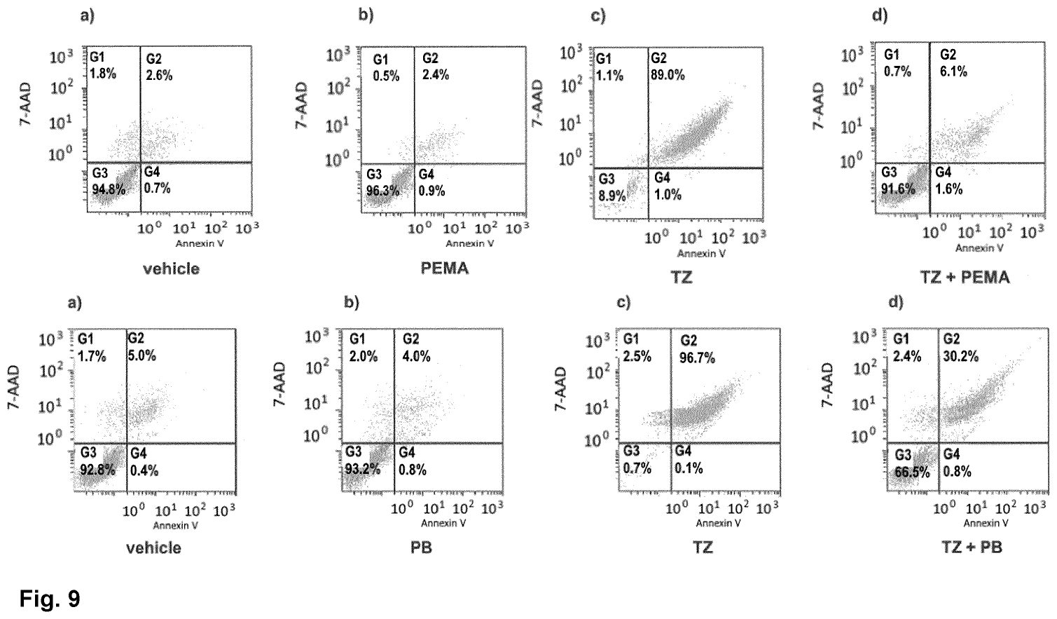

Results: As shown in panels (c) of FIG. 9, the combination of TNF-α and zVAD induces programmed cell death. This cell death is, as mentioned before (see FIGS. 1 and 8), RIPK1-dependent, and it can be blocked both with PEMA and PB, respectively as shown in panels (d). In summary, this experiment shows that the primidone metabolites PEMA and PB can block RIPK1-dependent necroptosis.

### Example 10: Primidone Protection Against SIRS

Hypothermia and morbidity induced by a high dose of TNF-α is considered in the literature to be a model for systemic inflammatory response syndrome (SIRS) (Moerke C, et al. (2019); Newton K, et al. (2014); Duprez L, et al. (2011)). All mice (8 weeks old) used in this experiment were on C57BL/6 background and age-, sex-, and weight-matched. Recombinant carrier-free murine TNF-α was obtained from R&D Systems (Bio-Techne, Wiesbaden, Germany). Each mouse received a single bolus of 1 mg murine TNF-α/kg body weight in a total volume of 200 ml phosphate-buffered saline, via the tail vein. In this setting, mice received 15 min before TNF-α application a single intraperitoneal (i.p.) injection (total volume per mouse was 200 μl) of either 2.5% DMSO in PBS (vehicle) or 10 mg primidone/kg body weight (as indicated). Thereafter, the animals (n=16 per group) were placed under permanent observation and survival was checked every 15 min. Survival is depicted in a Kaplan-Meier plot (*** p<0.001).

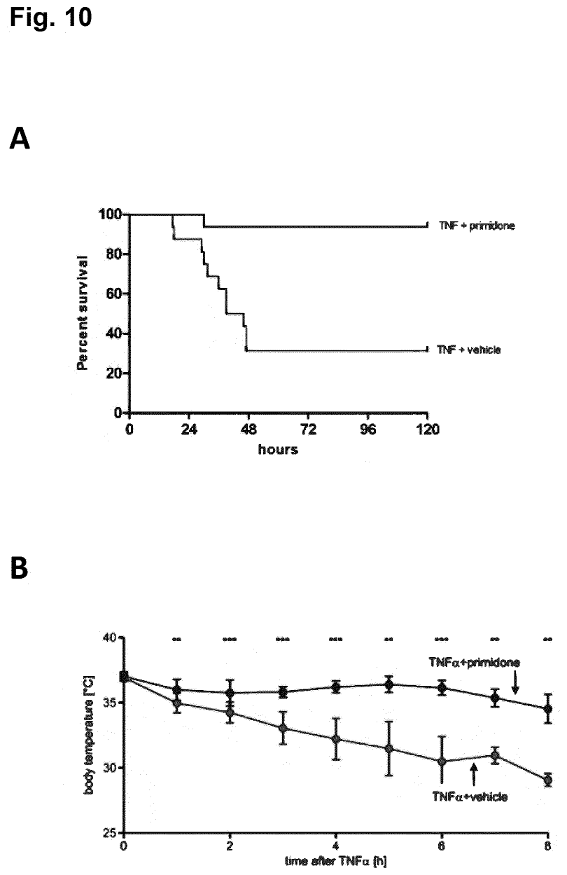

Results: The results are depicted in FIG. 10. It can be seen that mice that received primidone before TNF-α injection showed a significantly enhanced survival compared to the control group (FIG. 10A). It was also observed that the pharmacological inhibition of RIPK1 kinase by primidone significantly improves TNF-α-induced hypothermia (FIG. 10B). These results suggest that primidone is able to protect against the lethal consequences of SIRS and has therapeutic potential in patients suffering from a hyperinflammatory disease.

### Example 11: Kidney Ischemia-Reperfusion Injury (IRI)

IRI is a clinically highly relevant model, since it is an unavoidable consequence after kidney transplantation and contributes to acute kidney injuries in various contexts (Müller et al., 2017). To test whether primidone could be used to suppress pathophysiological RIPK1-mediated cell death in renal IRI, mice were provided with a drinking solution containing either primidone at 2.875 mM or vehicle in the regular drinking water for five days prior to ischemia reperfusion surgery until the end of the reperfusion phase. Induction of murine kidney IRI was performed via a midline abdominal incision and bilateral renal pedicle clamping for 37 min using microaneurysm clamps (Aesculap Inc., Center Valley, Pa., USA). Throughout the surgical procedure, mice were kept under isoflurane narcosis and the body temperature was maintained at 36° C. to 37° C. by continuous monitoring using a temperature-controlled self-regulated heating system (Fine Science Tools, Heidelberg, Germany). After removal of the clamps, reperfusion of the kidneys was confirmed visually before the abdomen was closed in two layers using standard 6-0 sutures. After 48 h reperfusion, the mice were sacrificed, blood samples were taken by retrobulbar punction and organs were collected for analysis.

For histology, kidney samples freshly obtained were fixed in 4.5% neutral-buffered formaldehyde and embedded in paraffin. Sections were dewaxed, rehydrated, and subjected to Masson trichrome staining according to routine protocols. Sections were dehydrated and mounted using DePeX mounting media (Serva). Stainings were evaluated in a blinded manner using a Leica Axiovert microscope and Axio Vision SE64 Rel 4.9. software (Leica Microsystems, Wetzlar, Germany). For data presentation, mild sharpening, contrast enhancement and gamma adjustment was performed. To analyze cell death of the tissue sections, a TdT-mediated dUTP nick end labelling (TUNEL) assay was performed using a fluorescence-based detection kit according to the manufacturer's instructions (G3250, Promega). Briefly, tissue sections were dewaxed, rehydrated, fixed in 4% paraformaldehyde and permeabilized with Proteinase K for 10 min at RT. Following this, the sections were equilibrated with the provided buffer for 10 min and labeled with the TdT reaction mix for 60 min at 37° C. in a humidified dark environment. To stop the labelling reactions, sections were incubated with the provided stopping buffer for 15 min at RT in the dark. The sections were then washed with PBS for 5 min. Finally, the sections were mounted with Shandon™ ImmuMount™ (Thermo Fisher Scientific). Fluorescence micrographs (data not shown) were acquired with a 20× and 40× objective magnifications using a standard fluorescein filter set to view the green fluorescence at 520 nm with a Leica Axiovert microscope and Axio Vision SE64 Rel 4.9 software. Quantification of TUNEL-positive cells was performed manually by two blinded observers by evaluating 8 randomly selected fields of view per slide.

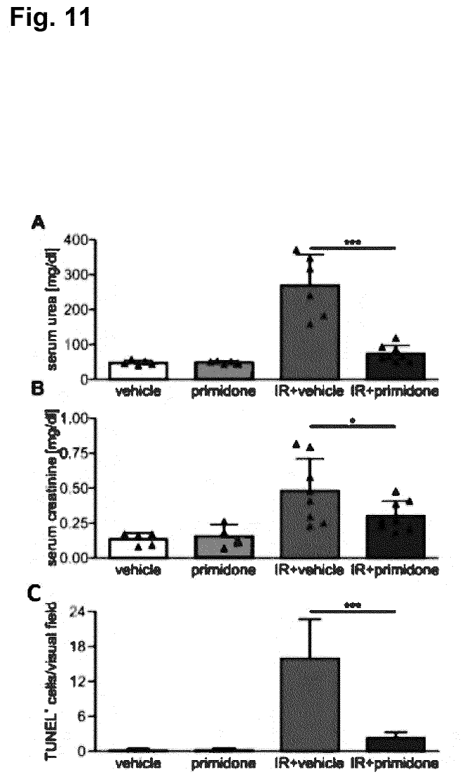

Results: The results are depicted in FIG. 11. Markers for the loss of kidney function (elevated serum concentrations of urea and creatinine) were significantly reduced 48 h after reperfusion in animals treated with primidone (FIGS. 11, A and B). This finding indicates the effectiveness and therapeutic potential of primidone for the treatment of complex diseases driven by RIPK1. A clear protective effect of primidone in this setting was also seen in Masson trichrome-stained histomicrographs of the renal outer medulla that display better preservation of tissue integrity when animals were treated with primidone (data not shown). In order to visualize the differences between the untreated and primidone treated animals in this model more prominently, strongly magnified images of these histologies were included. Therein, cellular debris and tubular necrosis of single cells are additionally marked in this extension. The corresponding TUNEL fluorescence assay showed a significant reduced number of cells undergoing regulated cell death in the primidone-treated cohort (FIG. 11C).

### Example 12: RIPK1 Activation in SARS-CoV-2 Patients

6 Patients who had been tested positive for SARS-CoV-2 within 48 h prior to sample acquisition and were hospitalized for displaying typical prominent clinical symptoms (fever, shortness of breath) were included. Ethical approval for this study was obtained from the local ethics committee (The Medical Faculty of the Christian-Albrechts-University of Kiel, Germany, AZ: D 495/20). All patients and controls participating in the study were informed of their rights as well as the risks and benefits of sample and data collection and gave informed written consent.

The case histories of the six patients who had been tested positive for SARS-CoV-2 were as follows:
SARS-CoV-2-positive-tested patient 1 (P1)
A 69-year-old male presented to our clinic after referral from his primary care physician because of fever up to 39° C. and progressive malaise. Symptoms had begun with a feeling of fever and a dry cough ten days before presentation. Additionally, he reported dysgeusia and night sweats, but no shortness of breath. He tested positive for SARS-CoV-2 infection upon arrival and a sample for this study was taken one day later. His only pre-existing medical condition was arterial hypertension. His breathing rate was 20 breaths per min, his heart rate was 70 beats per min, his blood pressure was 130/80 mmHg, and his body temperature was 38.3° C. Blood oxygen saturation was 96% under ambient air. Physical examination revealed fine crackles at the base of both lungs but was otherwise unremarkable. Small infiltrates at the base of the left lung were seen on chest X-rays. Laboratory results showed lymphocytopenia and increased inflammatory markers (C-reactive protein, IL-6, ferritin and D-dimer). Symptomatic treatment with acetaminophen was initiated. During the course of treatment, symptoms decreased gradually, but dysgeusia persisted. After two weeks of treatment and negative testing for SARS-CoV-2 he was discharged home.
SARS-CoV-2-positive-tested patient 2 (P2)
A 49-year-old male was transferred to our clinic from another hospital. He reported fevers of up to 40° C., progressive cough, and nasal discharge that had persisted for four days. He tested positive for SARS-CoV-2 infection at the other hospital one day before admission and repeat testing upon admission at our hospital showed borderline-positivity. A sample for this study was taken the next day. His pre-existing conditions were limited to diabetes mellitus type II. Upon presentation he complained of mild shortness of breath with a breathing rate of 19 per min but appeared otherwise healthy. His blood pressure was 145/80 mm Hg, his heart rate was 80 beats per min, and his body temperature was 38.9° C. Auscultation revealed fine crackles at the base of both lungs, but otherwise physical examination was unremarkable. Blood oxygen saturation was 93% under supplementation of 2 l/min oxygen flow through a nasal cannula. Infiltrates at the base of both lungs were seen on chest X-rays. Laboratory results were remarkable for increased markers of inflammation (C-reactive protein, IL-6, ferritin and D-dimer) and lymphopenia. Symptomatic treatment included acetaminophen and the intravenous application of crystalloid solutions. Five days after admission, the patient reported increasing shortness of breath. Arterial blood gas analysis showed hypoxemia with a partial pressure of 62 mmHg oxygen under supplementation of 3 l/min oxygen flow through a nasal cannula. Supplementation of oxygen was switched to a nonrebreather mask and increased to 4 l/min oxygen flow. Subsequently, the dyspnoea resolved over the course of five days, when supplementation of oxygen was ceased. The patient's condition improved under continued supportive care. After two weeks of treatment and negative testing for SARS-CoV-2 he was discharged home.
SARS-CoV-2-positive-tested patient 3 (P3)
A 64-year-old male was admitted to our hospital after collapsing at a nearby campground. He regained consciousness rapidly after emergency medicine technicians had diagnosed hypoglycemia (glucose 49 mg/dl, reference range 76-108 mg/dl) and intravenous glucose solution was applied. No other symptoms were noted, particularly no dysgeusia, cough, fever or dyspnea. The breathing rate was 16 breaths per min, the blood pressure was 142/76 mmHg, the heart rate was 78 beats per min, and the body temperature was 36.7° C. Physical examination was unremarkable except for moderate obesity and laboratory results as well as radiology studies showed no signs of infection or inflammation. SARS-CoV-2 infection was detected upon routine testing when the patient was admitted to the hospital and he remained isolated for two days. Pre-existing medical conditions were limited to diabetes mellitus type I and obesity. Since no further serious symptoms developed and the blood glucose levels had stabilized, the patient was released into quarantine at his home for 14 days 72 h after initial presentation with instructions to present to his primary care physician for a follow-up appointment.
SARS-CoV-2-positive-tested patient 4 (P4)
A 75-year-old male was initially evaluated in the emergency room of another hospital where he presented with a dry cough and severe difficulty breathing. Symptoms had gradually increased over the course of two days and a fever up to 39° C. had developed. He tested positive for SARS-CoV-2 infection upon arrival. Pre-existing medical conditions included diabetes mellitus type II, arterial hypertension, asthmatic lung diseases, hypothyroidism and benign prostatic hyperplasia. Physical examination showed a patient in apparent respiratory distress with crackles over the bases of both lungs. The breathing rate was 30 breaths per min, the blood pressure was 110/65 mmHg, the heart rate was 96 beats per min and the temperature was 38.4° C. Because the condition of the patient continued to deteriorate, he was placed on mechanical ventilation after successful intubation. A prolonged stay on the intensive care unit ensued. After six weeks of treatment, the patient was weaned and successfully extubated, but severe cough returned and a CT-scan of the chest showed radiological signs of COVID-19 without any hints of superinfection. Repeat testing showed continued positivity for SARS-CoV-2 and a sample for this study was obtained. A high-sensitivity troponin test showed a newly elevated troponin-I-level of 12,400 ng/l (reference range <45 ng/l), indicating acute myocardial damage. The EKG showed no pathological signs. Treatment with ASS 500 mg and heparin was initiated, but invasive diagnostics had not been conducted at the time of submission and neither myocardial infarction nor myocarditis had been ruled out.
SARS-CoV-2-positive-tested patient 5 (P5)
A 41-year-old female was admitted to our hospital because of joint pain, slight fevers and cough. She had recently been visited by her sister who was later tested positive for SARS-CoV-2-infection. Besides feeling very tired she did not notice any further symptoms, particularly no dyspnea or dysgeusia. The only pre-existing medical condition was minor thalassemia. Upon admission the breathing rate was 16 breaths per min, the blood pressure was 110/70 mmHg, the heart rate was 90 beats per min, and the body temperature was 37.7° C. Physical examination was unremarkable except for moderate obesity. Laboratory results showed a mild microcytic anemia, modest lymphopenia as well as moderately elevated C-reactive protein levels. An x-ray of the chest showed no signs of infection or inflammation. SARS-CoV-2-infection was detected when the patient was admitted to the hospital and a sample for this study was obtained. The patient remained in stable condition and was released to self-quarantine at home after two days of symptomatic treatment.
SARS-CoV-2-positive-tested patient 6 (P6)
A 32-year-old male was transferred from another hospital. He had previously been on vacation in Eastern Europe and developed severe difficulty breathing, fevers up to 39.5° C. and dysgeusia shortly after his return to Germany. He had tested positive for SARS-CoV-2 infection at the other hospital, where physical examination showed a patient in apparent respiratory distress with crackles over the bases of both lungs. The breathing rate was 32 breaths per min, the blood pressure was 124/74 mmHg, the heart rate was 98 beats per min and the temperature was 38.9° C. A CT-scan of the chest showed prominent infiltrates at the base of both lungs and laboratory markers of inflammation (C-reactive protein, IL-6, Ferritin and D-dimer) were markedly increased. Because of increasing respiratory distress a decision was made to transfer the patient to the intensive care unit, where repeat testing for SARS-CoV-2 was positive and a sample for this study was obtained. The breathing rate increased to 40 breaths per min and blood oxygen saturation decreased to 90%, but intubation was not deemed necessary and he improved under ventilation with continuous positive airway pressure and treatment with dexamethasone.
   Negative controls were collected from healthy individuals tested negative for SARS-CoV-2 infection. The controls did not show any signs of infection, in particular no cough, dysgeusia or sneezing, and no elevated inflammatory markers (C-reactive protein, IL-6, Ferritin and D-dimer). The following controls were used:
SARS-CoV-2-negative-tested control 1 (NC1): A 43-year-old male with no pre-existing medical conditions.
SARS-CoV-2 negative-tested control 2 (NC2): A 57-year-old female with no preexisting medical conditions.
SARS-CoV-2-negative-tested control 3 (NC3): A 41-year-old male with no pre-existing medical conditions.
SARS-CoV-2-negative-tested control 4 (NC4): A 79-year-old female with no preexisting medical conditions.
SARS-CoV-2-negative-tested control 5 (NC5): A 29-year-old male with no pre-existing medical conditions.
SARS-CoV-2-negative-tested control 6 (NC6): A 58-year-old male with no pre-existing medical conditions.
   From the patient and control individuals, cell smears were taken from the oropharyngeal epithelium, fixed in 4.5% formalin, blocked with horse serum, permeabilized with Triton X-100 and stained for phospho-RIPK1 using an anti-phospho-RIP1 antibody (44590, Cell Signaling Technology) and Alexa Fluor® 488-AffiniPure Donkey Anti-Rabbit IgG (711-545-152, Jackson ImmunoResearch Laboratories, West Grove, USA). Slides were mounted using ImmunoSelect® Antifading Mounting Medium with DAPI (SCR-038448, Dianova, Hamburg, Germany). Imaging was performed using a Zeiss Axio Imager Z1 fluorescence microscope and AxioVision Rel. 4.8 software (Carl Zeiss GmbH, Jena, Germany). Figures (not shown) were prepared using Fiji/ImageJ software (Schindelin et al., 2012). Grayscale images (not shown) were assigned the respective pseudocolor and channels were merged. 2× magnification insets were produced using the ImageJ macro “Zoom-in-Images-and-Stacks”. Mild background subtraction and gamma correction (gamma-value 0.9) was uniformly applied to display images for publication.

Results: The immunohistochemical analyses (data not shown) of pharyngeal epithelial cell samples from COVID-19 patients were positive for active, phosphorylated RIPK1. In complete contrast, no phospho-RIPK1-positive cells were apparent in the control samples from healthy individuals.

### LITERATURE

Cheng, Y. et al. Caspase inhibitor affords neuroprotection with delayed administration in a rat model of neonatal hypoxic-ischemic brain injury. J Clin Invest. 101(9), 1992-1999 (1998).
Cho, Y. S. et al. Phosphorylation-driven assembly of the RIP1-RIP3 complex regulates programmed necrosis and virus-induced inflammation. Cell 137, 1112-1123 (2009).
Degterev A. et al. Targeting RIPK1 for the treatment of human diseases. Proc Natl Acad Sci USA, 116(20), 9714-9722 (2019).
Eefing, F. et al. Role of apoptosis in reperfusion injury. Cardiovasc Res. 61(3), 414-426 (2004).
Do Y J eta al. A novel RIPK1 inhibitor that prevents retinal degeneration in a rat glaucoma model. Exp Cell Res, 359(1):30-38 (2017).
Duprez L, et al. RIP Kinase-Dependent Necrosis Drives Lethal Systemic Inflammatory Response Syndrome. Immunity. 35:908-918 (2011).
Falcon, C. et al. Exploiting Cell Death Pathways for Inducible Cell Elimination to Modulate Graft-versus-Host-Disease. Biomedicines, 5(2), 30 (2017).
Galluzzi L. et al. Necroptosis: Mechanisms and Relevance to Disease. Annu Rev Pathol, 12:103-130 (2017).
Gong Y. et al. The role of necroptosis in cancer biology and therapy. Mol Cancer. 18(1), 100 (2019).
Kanou T. et al. Inhibition of regulated necrosis attenuates receptor-interacting protein kinase 1-mediated ischemia-reperfusion injury after lung transplantation. J Heart Lung Transplant, 37(10), 1261-1270 (2018).
Khot W Y and Nadkar M Y The 2019 Novel Coronavirus Outbreak—A Global Threat. J. Assoc. Physicians India 68:67-71 (2020).
Linkermann A. et al. Rip1 (receptor-interacting protein kinase 1) mediates necroptosis and contributes to renal ischemia/reperfusion injury. Kidney Int, 81(8), 751-61 (2012).
Liu C. et al. Necroptosis: A novel manner of cell death, associated with stroke. Int J Mol Med, 41(2), 624-630 (2017)).
Moerke C, et al. Combined Knockout of RIPK3 and MLKL Reveals Unexpected Outcome in Tissue Injury and Inflammation. Front Cell Dev. Biol. 7:19 (2019).
Müller, T. et al. Necroptosis and ferroptosis are alternative cell death pathways that operate in acute kidney failure. Cell. Mol. Life Sci. 74:3631-3645 (2017).
Newton K, et al. Activity of Protein Kinase RIPK3 Determines whether Cells Die by Necroptosis or Apoptosis. Science 343:1357-1360 (2014).
Oerlemans M I, et al. Inhibition of RIP1-dependent necrosis prevents adverse cardiac remodeling after myocardial ischemia-reperfusion in vivo. Basic Res Cardiol, 107(4), 270 (2012).
Pan L, et al. Activation of necroptosis in a rat model of acute respiratory distress syndrome induced by oleic acid. Acta Physiologica Sinica, 68(5): 661-668 (2016).
Pavlosky A. et al. RIPK3-mediated necroptosis regulates cardiac allograft rejection. Am J Transplant, 14(8), 1778-90 (2014).
Rickard, J. A. et al. RIPK1 regulates RIPK3-MLKL-driven systemic inflammation and emergency hematopoiesis. Cell 157, 1175-1188 (2014).
Sato K. et al. Receptor interacting protein kinase-mediated necrosis contributes to cone and rod photoreceptor degeneration in the retina lacking interphotoreceptor retinoid-binding protein. J Neurosci, 33(44), 17458-68 (2013).
Schindelin J. et al. Fiji: an open-source platform for biological-image analysis. Nat. Methods. 9:676-682 (2012).
Shi, S. et al. Necroptotic Cell Death in Liver Transplantation and Underlying Diseases: Mechanisms and Clinical Perspective. Liver Transpl., 25(7), 1091-1104 (2019).
Sun, L. et al. Mixed lineage kinase domain-like protein mediates necrosis signaling downstream of RIP3 kinase. Cell 148, 213-227 (2012).
Takemoto K. et al. Necrostatin-1 protects against reactive oxygen species (ROS)-induced hepatotoxicity in acetaminophen-induced acute liver failure. FEBS Open Bio, 4, 777-87 (2014).
Zhang, T. et al. CaMKII is a RIP3 substrate mediating ischemia- and oxidative stress-induced myocardial necroptosis. Nat Med. 22(2), 175-82 (2016).
Zhang, S. et al. Necroptosis in neurodegenerative diseases: a potential therapeutic target. Cell Death Dis., 8(6):e2905 (2017).
Yuan, J. et al. Necroptosis and RIPK1-mediated neuroinflammation in CNS diseases. Nat Rev Neurosci, 20(1):19-33, (2019).
Zelic M. et al. RIP kinase 1-dependent endothelial necroptosis underlies systemic inflammatory response syndrome. J Clin Invest, 128(5), 2064-2075 (2018).

1. Primidone or a pharmaceutically acceptable active metabolite, derivative, salt or solvate thereof for use in a method of treating a disease that involves a pathologic level of RIPK1-dependent cell death, wherein said disease is a reperfusion injury disease, a systemic inflammatory disease, a neurodegenerative disease, an autoimmune disease, or graft-versus-host disease, wherein the pharmaceutically acceptable active metabolite is phenobarbital.

2. (canceled)

3. Primidone or a pharmaceutically acceptable active metabolite, derivative, salt or solvate thereof for use in a method of claim 1, wherein the reperfusion injury disease is selected from myocardial infarction, stroke, acute kidney failure, and acute liver failure.

4. Primidone or a pharmaceutically acceptable active metabolite, derivative, salt or solvate thereof for use in a method of claim 1, wherein the neurodegenerative disease is selected from Alzheimer's disease, Parkinson's disease, Huntington's disease, and amyotrophic lateral sclerosis (ALS), traumatic brain injury, and multiple sclerosis (MS).

5. Primidone or a pharmaceutically acceptable active metabolite, derivative, salt or solvate thereof for use in a method of claim 1, wherein the autoimmune disease is selected from ulcerative colitis, Crohn's disease, rheumatoid arthritis, autoimmune cardiomyopathy, autoimmune hepatitis, lupus erythematosus, Graves' disease, Guillain-Barré syndrome (GBS), Hashimoto's thyroiditis, idiopathic thrombocytopenic purpura, juvenile idiopathic arthritis, myasthenia gravis, pemphigus vulgaris, psoriasis, Reiter's syndrome, scleroderma, Sjögren's syndrome, vasculitis, vitiligo, and Wegener's granulomatosis.

6. Primidone or a pharmaceutically acceptable active metabolite, derivative, salt or solvate thereof for use in a method of claim 1, wherein the systemic inflammatory disease is selected from sepsis and systemic inflammatory response syndrome (SIRS).

7. Pharmaceutical composition comprising primidone or a pharmaceutically acceptable active metabolite, derivative, salt or solvate thereof for use in a method of treating a disease or condition that involves a pathologic level of RIPK1-dependent cell death, wherein said disease is a reperfusion injury disease, a systemic inflammatory disease, a neurodegenerative disease, an autoimmune disease, or graft-versus-host disease, wherein the pharmaceutically acceptable active metabolite is phenobarbital.

8. (canceled)

9. Pharmaceutical composition for use in a method of claim 7, wherein the reperfusion injury disease is selected from myocardial infarction, stroke, acute kidney failure, and acute liver failure.

10. Pharmaceutical composition for use in a method of claim 7, wherein the neurodegenerative disease is selected from Alzheimer's disease, Parkinson's disease, Huntington's disease, and amyotrophic lateral sclerosis (ALS), traumatic brain injury, and multiple sclerosis (MS).

11. Pharmaceutical composition for use in a method of claim 7, wherein the autoimmune disease is selected from ulcerative colitis, Crohn's disease, rheumatoid arthritis, autoimmune cardiomyopathy, autoimmune hepatitis, lupus erythematosus, Graves' disease, Guillain-Barré syndrome (GBS), Hashimoto's thyroiditis, idiopathic thrombocytopenic purpura, juvenile idiopathic arthritis, myasthenia gravis, pemphigus vulgaris, psoriasis, Reiter's syndrome, scleroderma, Sjögren's syndrome, vasculitis, vitiligo, and Wegener's granulomatosis.

12. Pharmaceutical composition for use in a method of claim 7, wherein the systemic inflammatory disease is selected from sepsis and systemic inflammatory response syndrome (SIRS).

13. Pharmaceutical composition for use in a method of claim 7, wherein said composition is formulated for being administered by injection.

14. Pharmaceutical composition for use in a method of claim 7, wherein said composition comprises at least one additional inhibitor of apoptosis or necroptosis.

15. Pharmaceutical composition for use in a method of claim 7, wherein primidone or the pharmaceutically acceptable active metabolite, derivative, salt or solvate thereof is administered daily in an amount of comprise 1-50 mg per kilogram body weight of the patient.
